(19) United States
(12) Patent Application Publication
(10) Pub. No.: US 2007/0293904 A1
(43) Pub. Date: Dec. 20, 2007

(54) Self-powered resonant leadless pacemaker

(76) Inventors: Daniel Gelbart, Vancouver (CA); Samuel Victor Lichtenstein, Vancouver (CA)

Correspondence Address: SEED INTELLECTUAL PROPERTY LAW GROUP PLLC, 701 FIFTH AVE, SUITE 5400, SEATTLE, WA, 98104, US

(21) Appl. No.: 11/455,827

(22) Filed: Jun. 20, 2006

(51) Int. Cl. A61N 1/00

(52) U.S. Cl. 607/35

(57) ABSTRACT

A self-powered medical device, for example a pacemaker uses the variations of blood pressure inside the heart or a major artery to create a mechanical resonance in an electromagnetic or piezoelectric generator. The resonance extends the time power is generated during the cardiac cycle. The pressure variations compress a bellows carrying the resonant generator. The inside of the bellows may be evacuated to a partial or full vacuum, and a spring restores the bellows to the desired equilibrium point, acting against the blood pressure. The current pulses are stored in a capacitor. Eliminating the battery allows dramatic miniaturization of the medical device to the point it can be implanted at the point of desired stimulation via a catheter.

### TECHNICAL FIELD

The disclosure relates to self-powered medical devices inside the body and in particular to cardiac pacemakers.

### DESCRIPTION OF THE RELATED ART

Cardiac pacemakers are well known, however they have three major shortcomings:

A. They require major surgery to install and to replace.

B. They have a limited lifetime because of the battery.

C. They require running leads from pacemaker to the heart chambers. The leads reduce the reliability of the device and make replacement difficult.

There were many prior attempts to overcome the battery problem by using rechargeable batteries (charged by induction) or electrical energy generated inside the body. To date these attempts were not successful. Rechargeable batteries do not have a longer life than primary batteries at the low power drain of pacemakers (10-50 microwatts), and implanted devices that generate electrical energy from the motion of the heart were not significantly smaller than the batteries and still required leads. Most reported devices did not generate a sufficient amount of energy.

The main reason prior devices did not generate sufficient energy is due to the fact that the motion of the heart is not of constant velocity or acceleration, therefore the voltage generated varies widely over a single cardiac cycle. This requires a capacitor to average out the voltage. The device can only generate energy when the generated (i.e., induced) voltage exceeds the voltage of the capacitor, causing current to flow into the capacitor. This happens for only a short fraction of the cardiac cycle, and is the main reason for the low output of prior devices. In order to improve the situation prior devices tried to increase the magnitude of the induced voltage, by mechanical gearing and snap action devices, to deliver more power during the interval when the current flows into the capacitor. Other prior devices tried to increase the duration of the current flow by mechanically resonating the device creating the induced voltage to generate a more continuous flow of current. It was found by the present inventors that neither method is sufficient for generating the amount of power a pacemaker requires out of a small volume.

### BRIEF SUMMARY

The desired volume of a pacemaker is below 3 cubic centimeters, and ideally below 2 cubic centimeters. Such a volume allows implanting the pacemaker directly into the heart via a catheter percutaneously. A percutaneous procedure is much superior to conventional surgery, as is any minimally invasive surgery compared to conventional surgery. Percutaneous delivery also requires the pacemaker to have a particular form factor, typically an elongated cylinder under 10 mm in diameter.

The present disclosure provides a device that can generate a significant amount of power (beyond the need of a standard pacemaker) and be delivered percutaneously. It was found that a device that increases the natural velocity or acceleration of the heart muscles (to increase the induced voltage) and at the same time extends the duration of the current, by using a low loss mechanical resonator, can provide sufficient power in such a small volume.

One simple way to increase the speed of movement created by the heart muscles is to power the device from the blood pressure and not directly from the muscle movement. It is well known that the blood pressure inside the heart, and in particular inside the left ventricle, rises and falls very fast. A bellows responding to this rapid change in blood pressure will move significantly faster than the wall of the ventricle. The reason is that the wall area is much larger than the area of the bellows, so a small movement of the wall creates a large change in volume, causing the bellows to move a significant amount. Prior attempts to use this principle, such as US RE30,366, fails to take into account the very low pressure differentials inside the heart in comparison to atmospheric pressure, thus the energy extracted will be only a small fraction of the estimated power. For example, RE30,366 estimates that the 20 mmHg pressure pulse of the right ventricle will move the transducer 1 mm, generating 130 micro joule of energy (page 8 line 32) while the actual number is only a small fraction of this number. The reason is that any movement of the bellows will increase the air pressure inside the device. In a 1 cm long enclosure, even if the enclosure was completely empty, the movement will only be: 10 mm×20 mmHg/760 mmHg=0.26 mm. When enclosure is filled with the necessary pacemaker electronics, movement is further reduced. In order to achieve high efficiency the transducer has to avoid the increase in internal air (or gas) pressure when its volume is changing. The present embodiments allow movements of several millimeters from very low pressure changes, with corresponding increases in output power.

A second shortcoming of prior attempts is failing to take into account the effect of high air pressure at high altitudes or inside airplane cabins. The pressure inside an airplane cabin is about 200 mmHg lower than at sea level. This is about 10 times the magnitude of the pressure pulse in the right ventricle. Any device designed to operate on a pressure differential of 20 mmHg and does not take into account an external pressure differential of 200 mmHg is of limited use. Prior attempts based on blood pressure also fail to use a resonator to extend the duration of current flow. Because of these reasons the reported output of small size prior art devices is under 10 uW, regardless of type of generator. US RE30,366, as well as U.S. Pat. No. 3,554,199 (page 2 lines 15-18) mention the possibility of an inertial device (i.e., not operated by blood pressure but by the effect of acceleration on a mass) made to resonate with the heart rate. Since the heart rate is about 1 Hz, the induced voltage, which is proportional to the resonant frequency, will be very low, unless the amplitude of the motion is large. The small dimensions of a catheter delivered device rule out a large motion amplitude, thus the resonant frequency has to be significantly higher than the heart rate. Prior devices cannot induce such a resonance as the accelerations involved in the heart wall motion are too low. If one tries to use a higher resonant frequency the amplitude of the resonance will be very low and again the induced voltage will be very low. This can be seen from the following calculation: Assume the acceleration of the heart wall is “a” and a mass “m” is mounted on a spring having a spring constant “k”. The resonant frequency is proportional to the square root of k/m. The induced voltage is proportional to the velocity, which is proportional to the product of the amplitude times the frequency. The initial amplitude “A” is given by the force, F=ma acting on the spring constant “k”: A=ma/k. The voltage is proportional to: V˜frequency×amplitude˜(k/m)^{̂}0.5×ma/k˜a×(m/k)^{̂}0.5 .

This calculation shows that for an inertial device, the voltage is proportional to the input acceleration. To increase the voltage means for increasing the heart wall acceleration are required. The present embodiments provide such means, but only when such means are combined with a suitable mechanical resonance sufficient power will be generated. Prior attempts fail to combine such “snap action” (to increase voltage) with mechanical resonance (to increase duration of current flow).

In one aspect, a self-powered pacemaker of such small size that it can be implanted at the point of the desired stimulation, thus requiring no leads. The small size also allows percutaneous implantation and replacement, as the device is small enough to fit through the catheters currently used in percutaneous cardiac surgery. If desired, the device can be used with conventional pacing leads. The device can also be used simply as an electrical energy generator inside the body. It can be placed in the heart or in any major artery to supply electricity for devices other than pacemakers, for example de-fibrillators, drug delivery devices, brain stimulators etc. A device having a volume of about two cubic centimeters can supply over approximately 33 microwatts continuously. The theoretical possible power output from a one cubic centimeter device placed in the left ventricle of the heart and powered by the blood pressure variation is about 10 mW, thus less than 1% efficiency is required to power a pacemaker. In another aspect, a device may be tolerant to large changes in ambient air pressure without electrical output being affected. In yet another aspect, a very reliable device is not subject to internal wear, by avoiding any internal friction and basing all motions on flexure instead of bearings.

In at least one embodiment, a device uses the variations of blood pressure inside the heart, or a major artery, to create a periodic change in the magnetic flux inside a coil by resonating a mass-spring system. Typically the pressure variations compress a bellows carrying a magnet resonating inside a coil. The inside of the bellows can be evacuated to a partial or full vacuum, and a spring restores the bellows to the desired equilibrium point, acting against the blood and atmospheric pressure. The electrical pulses may be stored in a capacitor, and used to power a pacemaker or other devices. Since most of the volume of a pacemaker is the battery, eliminating the battery allows dramatic miniaturization of the pacemaker, to the point it can be implanted at the point of desired stimulation. There is no other mechanical coupling to the heart motion except via the changes in blood pressure. This minimizes the interference with the operation of the heart. The compressibility of the device volume with increased pressure is actually an advantage, as it reduces the blood pressure peaks. The device allows for the ambient air pressure to change by allowing the bellows to change length without affecting electrical output.

In at least one embodiment, a resonant electrical generator is inertially excited by the heart wall movement. In order to increase the acceleration powering the resonance, the motion is made highly non-linear inside the device by using motion limiters or a snap action spring.

### BRIEF DESCRIPTION OF THE SEVERAL VIEWS OF THE DRAWINGS

FIG. 1 is an isometric exploded view of a medical device according to one illustrated embodiment, with a bellows removed from a base to show the internal parts.

FIG. 2 is a longitudinal cross-sectional view of the medical device of FIG. 1 implanted in a wall of a heart, according to one illustrated embodiment.

FIG. 3A is an isometric view of a piezoelectric resonant generator for use in the medical device of FIG. 1, according to one illustrated embodiment.

FIG. 3B is an isometric view of an electromagnetic resonant generator for use in the medical device of FIG. 1, according to another illustrated embodiment.

FIG. 4 is an electrical schematic diagram of a circuit for use in the medical of FIG. 1, according to one illustrated embodiment.

FIG. 5 is a longitudinal cross-sectional view of a medical device according to an alternate illustrated embodiment, implanted in a portion of a body.

FIG. 6 is a graph of the variations of blood pressure and voltage produced by the medical device of FIG. 1, according to one exemplary embodiment.

FIG. 7 is a graph of the ventricular wall velocity, frame velocity and voltage produced by the medical device of FIG. 5, according to another exemplary embodiment.

FIG. 8 is a cross-sectional view of a catheter in use to implant a medical device in a portion of a body, according to one illustrated embodiment.

FIG. 9 is a cross-sectional view of a medical device with a detachable magnetic base implantable in a portion of the body, according to yet another illustrated embodiment.

### DETAILED DESCRIPTION

In the drawings, identical reference numbers identify similar elements or acts. The sizes and relative positions of elements in the drawingis are not necessarily drawn to scale. For example. the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used in this specification and the appended claims. the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

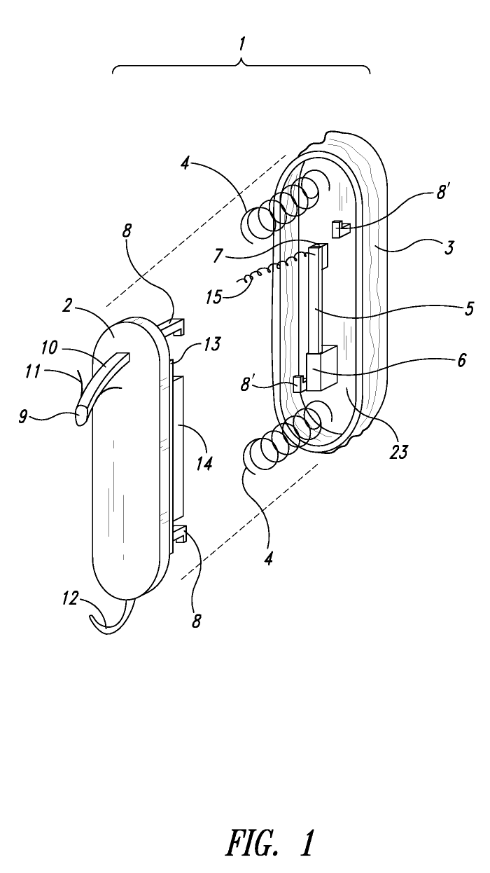

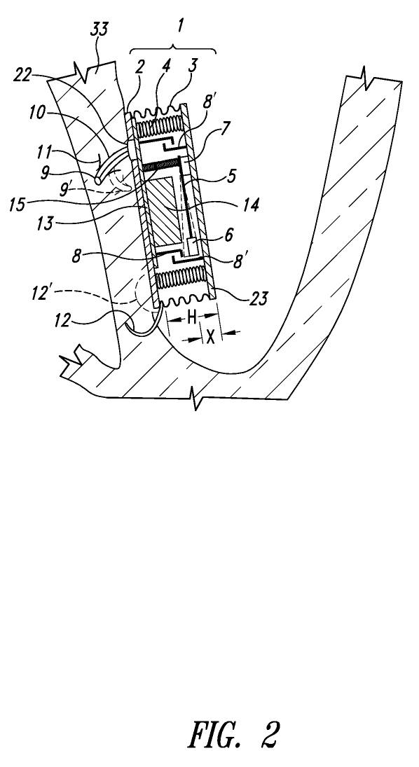

A medical device uses a resonant generator, either of the piezoelectric type or the electromagnetic type, to extend the portion of the cardiac cycle in which energy is transferred to a storage capacitor. As the medical device, for example a pacemaker 1, needs to fit through a catheter, the room allowed for the motion is very limited, on the order of a few millimeters. In order to generate sufficient power, the frequency of the resonance has to be significantly higher than the natural heart rate, as the generated voltage is proportional to the velocity which is proportional to the product of frequency and amplitude. The desired frequency range for the resonance is 10 Hz-100 Hz. The Fourier spectrum of the heart muscle motion contains very little energy at this range, therefore a way of increasing the acceleration is required before a resonant generator can be efficiently driven. Two approaches to increasing acceleration are disclosed: using the blood pressure as a source of motion and using a non-linear transformation of the heart wall motion to generate high frequencies. The Fourier spectrum of the ventricular blood pressure profile contains significantly more high frequencies than the spectrum of the ventricular wall motion. A second advantage of generating higher frequencies for driving the resonant generator is the wide variations in heart rate. Such wide variations prevent the use of a highly tuned resonant circuit. On the other hand, when many high order harmonics are generated from the basic motion there will always be a harmonic which matches the resonant frequency, as the spectral spacing of the harmonics is about 1 Hz (the heart rate) while the bandwidth of the resonant generator can be made to be at least 1 Hz and still have a sharp resonant. By the way of example, a 30 Hz resonator with a 1 Hz bandwidth will keep resonating with a significant amplitude throughout the cardiac cycle. In contrast, a resonant generator tuned to the heart rate of 1 Hz and having a 1 Hz bandwidth will not resonate at all if the heart rate goes up to 2 Hz, as a 2 Hz waveform has no Fourier component at 1 Hz. At least a first embodiment uses the blood pressure pulse inside the ventricle (either right or left) to drive the resonant generator. Referring now to FIG. 1, a medical device, for example a pacemaker 1a comprises of a rigid base 2, rigid cover 23, bellows 3, resonant mass 6, piezoelectric generator 5 (also acting as spring) anchored to cover 23 via mount 7, and electronics module 14 mounted on electronics board 13. The inside of bellows 3 is partially or fully evacuated and springs 4 are used to restore the position of bellows 3 against atmospheric pressure. Motion limiters 8 and 8′ prevent damage to bellows when transported under low air pressure conditions, such as when shipped as air cargo. Flexible lead 15 connects the output of generator 5 to board 13. The body of the pacemaker 1 forms the other lead. Referring now to FIG. 2, pacemaker 1a is attached the heart wall 33 using flexible electrodes 9 and 12, which are elastically deformed to pull pacemaker 1a towards wall 33 and at the same time serve as pacing electrodes. When pacemaker 1a is located inside a heart chamber or major artery, the blood pressure acts on cover 23 and compresses bellows 3. Since blood pressure changes with cardiac movement, cover 23 moves by an amount X between position shown as H and position shown by broken line. Electrode 9 is insulated from base 2 using hermetic seal 22, typically a glass-to-metal seal. Part of the electrode is covered by insulation 10 and has retention barbs 11. Bellows 3 is made of metal such as nickel or stainless steel and is welded to base 2, typically by electron-beam welding. The significance of the all metal construction of the enclosure and the hermetic sealing goes beyond the need for reliability. The operation of the device requires that the hermetic seal will be preserved indefinitely, as explained later on. Electronics 14 contains standard pacemaker circuitry and will not be detailed here as it is well known in the art.

In non-pacemaker embodiments, the electronics 14 may include other suitable circuitry, for example circuitry suitable for use with defibrillators, drug delivery devices, brain stimulators, etc. Electrodes 9 and 12 serve both to anchor the pacemaker to the interior of the heart as well as pacing electrodes. Not all electrodes need to be active, some can be used simply for mechanical anchoring and have no electrical function. Electrodes 9 and 12 can be used as an antenna when the pacemaker 1 communicates with external programming devices, or as electrical leads to charge the energy storage capacitor before installation in heart. All standard modes of pacing can be implemented by choosing the number, size and placement of electrodes. In the preferred embodiment the electrodes are made of flexible material such as Nitinol in order to elastically hold pacemaker to the tissue and to be able to flex them when inserted via catheter. The relaxed shape of electrodes 9 and 12 is shown by broken lines 9′ and 12′, respectively. In FIG. 2 the electrical generator is a piezoelectric bimorph, but it can easily be replaced by a magnet and coil as shown later on.

In order to make the size of the device as small as possible, the unused internal air space is minimized. This creates a problem, as internal air is compressed when bellows 3 is compressed. The internal air pressure rises as H/(H−X) for an empty case, and much faster if some of the airspace is used or occupied. By the way of example, if H in FIG. 2 is 6 mm and half of the internal space is used, leaving an effective H of 3 mm, a blood pressure pulse of 20 mm Hg, as is typical of the right ventricle, will move cover 5 only: x=3 mm×20 mmHg/760 mmHg=0.08 mm. This is insufficient to power pacemaker circuitry. It was found experimentally that a movement of over 1 mm is desired, 2 mm being preferred, in a miniature device than can be delivered via a catheter. In the previous example a movement of 1 mm will require a pressure of about 250 mmHg and a movement of 3 mm is not possible, as it will require infinite pressure (since the 3 mm airspace will need to compress to zero volume). The problem is solved by fully or partially evacuating the inside of the pacemaker 1 and providing a spring 4 which is always partially compressed. Such a spring restores the position of cover 23 in FIG. 2 to height H, allowing blood pressure to compress it by a distance X. It is desired to choose a spring with a very low spring constant k and a large preload, as seen from the following calculation: Initial length of spring 4 is L, compressed length is H. Force is k(L−H) based on the well-known spring formula. The bellows 3 is considered part of the spring constant, or can replace the spring altogether.

Effective area of bellows 3 is A (the effective area is derived from the volume change for a given movement X, V=AX), and blood pressure changes from a low of P1 to a high of P2 (for example, from 5 to 25 mmHg in the right ventricle).

The values of k and L are derived from the following equations:

A(P1+760 mmHg)=k(L−H)

A(P2+760 mmHg)=k(L−H+X)

By the way of example (substituting 13.6 gm/cm² for every 10 mmHg):

H=6 mm, X=2 mm, A=2 cm^{2,} P1=5 mm Hg (6.8 gm/cm²), P2=25 mmHg (34 gm/cm²)

k(L−6)=2(5+760 mmHg)=2081 gm

k(L−6+2)=2(25+760 mmHg)=2135

Solving for k and L gives k=approx 27 gm/mm and L=approx 83 mm.

The reason why additional springs 4 are sometimes required is the need to make the wall of the bellows very thin to achieve practically infinite fatigue life. It maybe important to keep the deformation of the bellows below 30% of its elastic range. This requires a very thin-walled bellows, which may not have a sufficient k.

If some air is left behind inside the device, assuming a partial pressure p, expressed as a fraction of atmospheric pressure (p=1 at 760 mmHg), the equations become:

A(P1+760 mmHg−p760 mmHg)=k(L−H)

A(P2+760 mmHg−p760 mmHg.H/(H−X)=k(L−H+X)

The term (H−X)/H is the increase in p as the volume decreases.

It is clear from the equations that p can only be a very small number before the term pH(H−X) will overpower the effect of the blood pressure, limiting the travel to a very short distance.

Since atmospheric pressure changes can be larger than changes in blood pressure during a cardiac cycle, bellows 3 has to allow movement for atmospheric pressure of about 200 mmHg. For example, the pressure in an airplane cabin can be as low as 560 mmHg. As can be seen, there is a trade-off between the overall height of the device H, the travel X and the atmospheric pressure variations it can operate under. If a small H is desired, a large k spring will have to be used and a small X will result. The power generated is proportional to X. This also means that a device placed in the left atrium or major artery will be more compact than a device placed in the right one, as the blood pressure pulse is 4-5 times larger, allowing k to be larger by the same amount. The travel limiters 8 and 8′ have to allow the full range of about 500-800 mmHg, however the device can be compressed to the H−X height for insertion through the catheter.

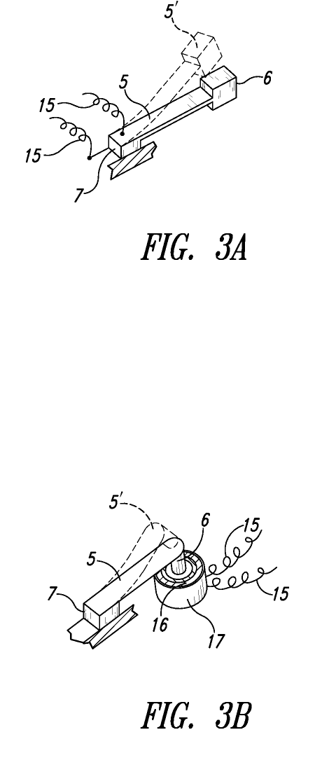

While the previous description shows a piezoelectric generator, FIG. 3A and FIG. 3B show the easy interchangeability of piezoelectric and electromagnetic generators. In FIG. 3B a piezoelectric bimorph 5, typically made of PZT, acts as a spring for mass 6. The other end is rigidly anchored by base 7. Flexible leads 15 are used to carry the current. The fully flexed position is shown in broken line 5′. In FIG. 3A one side of the bimorph is grounded. In FIG. 3B, the generator comprises of leaf spring 5 with a rare-earth magnet 6 acting as a mass. When the mass and spring resonate, the flux from magnet 6 that intersects coil 16 is changing and an induced current flows via leads 15. Ferromagnetic sleeve 17 slightly improves performance. A second coil, identical to coil 16 can be added above magnet 6 in order to increase the output, as there will be a changing magnetic field above and below moving magnet 6. A coil moving in a stationary magnetic field can be used as well. This configuration is preferred when the pacemaker 1 contains magnetic or highly conductive components, as they increase damping of the moving magnet. Also, a moving magnet can induce undesirable voltages in highly sensitive parts of the circuitry.

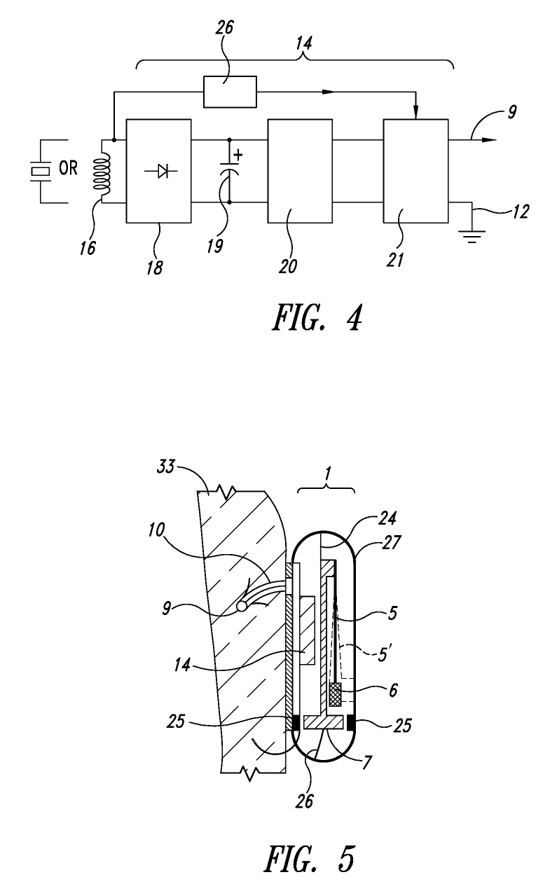

FIG. 4 is an electrical schematic of the pacemaker 1. The pulses at the output of coil 16 (or piezoelectric bimorph) are rectified by bridge rectifier 18, charging capacitor 19 and powering a DC-to-DC converter or simply a voltage regulator 20. In order to use a simple regulator rather than a DC-to-DC converter, coil 16 is wound with very fine magnet wire to generate directly a voltage higher than the voltage required by the pacemaker electronics, or a high output voltage piezoelectric bimorph is used. The rest of the circuitry may be a conventional pacemaker 21, pacing the heart via electrodes 9 and 12. Capacitor 19 can be a tantalum capacitor (to allow reserve power for a few minutes) or a super-capacitor. A super-capacitor will power a pacemaker for many hours without any charging current. A rechargeable battery can be used for even larger capacity, however batteries have a shorter life than dry tantalum capacitors, which have no lifetime limit. It may be desired to supply the pacemaker electronics 21 with information about blood pressure or heart wall movement. Since the voltage in coil 16 is proportional to the derivative of the pressure or wall movement, it is simple to integrate this voltage and recreate the pressure or movement waveform. This is shown symbolically by integrator 26. The integration can be performed numerically, of course, by a computer controlling the pacemaker functions. It will be appreciated that a generator responding to blood pressure, as shown here, will also respond to the movement of the whole unit as the resonant generator also acts as an accelerometer. While this component is smaller than the acceleration caused by the blood pressure, it can be used for sensing and synchronization, as explained later on.

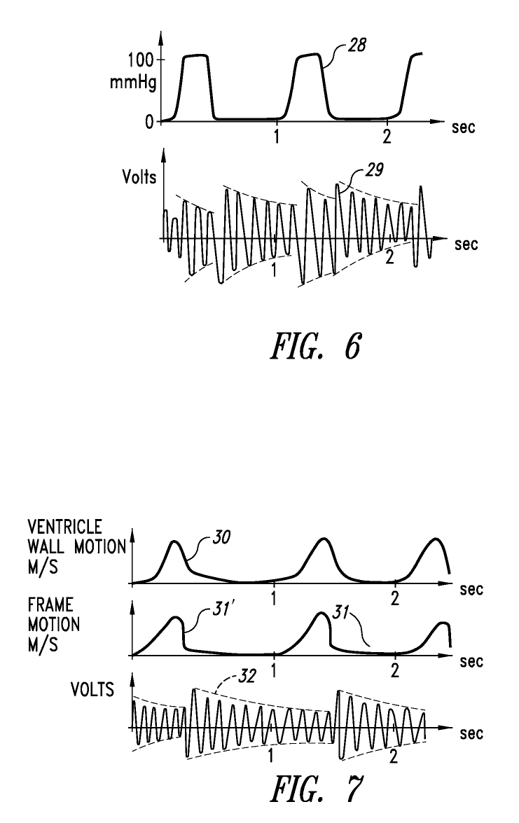

FIG. 5 shows an alternate embodiment of a resonant generator powered medical device in the form of a pacemaker 1b. The generating elements, 5 and 6, are identical to the previous embodiment. The excitation of the oscillations is different. The pacemaker 1b is anchored to the inside wall 33 of the left or right ventricle, or any other part of the human body that is constantly moving. Since the acceleration of heart wall 33 is not sufficient to excite resonance in generator 5 at the desired frequency (typically 10-100 Hz), an abrupt change in velocity is required to generate a higher acceleration. This is done by mounting generator 5 on a mounting frame 7 which is suspended by a soft spring 24 from pacemaker housing 27. When frame 7 is moved, in response to movement in pacemaker 1b, it will come to an abrupt stop when hitting stops 25. The resulting high acceleration will excite generator 5 into resonance. A different embodiment adds “snap action” to frame 7 by adding another spring 26. Frame 7 has two stable positions now, touching either the left hand or the right hand stops 25. As heart wall 33 moves, frame 7 is snapping between these two positions, increasing acceleration. The natural frequency of frame 7 versus housing 27 should be quite low, in the order of 1 Hz, to maximize the movement of frame 7 relative to housing 27. As in previous embodiments, generator 5 can be a piezoelectric bimorph, a moving magnet or a moving coil. Variable reluctance generators are less desired as they cause a larger damping. In this embodiment there is no need to evacuate enclosure 27.

FIG. 6 shows typical waveforms for a blood pressure activated device such as that of FIG. 1. Graph 28 shows the left ventricle blood pressure and graph 29 showed the damped resonance of generator 5 and mass 6. It should be noted that a very low damping will produce more power but the power will have stronger variations with changing heart rates, as the Fourier components of the movement spectrum will not always line up well with the narrow excitation spectrum. A higher damping will have a wider excitation spectrum and more stable output. Graph 29 shows a typical waveform with correct damping. The reason for the low damping is the low overall electrical efficiency of the generator (a few percent). Clearly, any efficient generator will be highly damped.

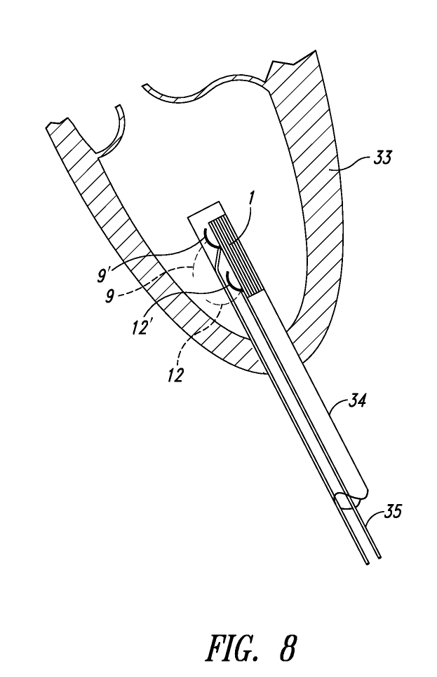

FIG. 7 shows the waveforms in the pacemaker of FIG. 5. Graph 30 shows the ventricular wall velocity, graph 31 shows the frame velocity, the sudden jump 31′ happens when the frame hits the stops 25. Graph 32 shows the induced voltage of the resonant generator.

FIG. 8 depicts the implantation of a medical device such as the pacemaker 1 in a typical minimally invasive, or percutaneous, procedure. The pacemaker 1 is delivered into the ventricle via catheter 34. Wires 35 are used to force the flexible electrodes 9′ and 12′ into positions 9 and 12 after pacemaker 1 is pushed out of catheter. At position 9 and 12 the electrodes are pushed into ventricular wall 33 and released. Other catheter based procedures can be used, not requiring piercing a hole in the heart, by entering through the aorta or other major blood vessels. After a while the pacemaker 1 may become covered with endocardium, which is sufficiently flexible not to interfere with the device operation. If this is not desired, the outside of the pacemaker 1 can be coated with a drug eluting coating or a hydrophobic coating such as thin silicone, or fluorocarbon. While the preferred embodiment is to locate the device inside the arterial blood system, the device can be located outside any major artery, with the cover 27 pressed to the artery wall, and responding to the wall moving with the pressure pulse. Electrode 9 and 12 can be replaced by leads or any other device. All the advantages, such as low operating pressure, are maintained regardless of device being inside or outside the artery wall.

By the way of example, bellows 3 is a 30 mm long×10 mm wide×8 mm high custom-made bellows made of nickel available from the Servometer Corporation (www.servometer.com). Magnet 6 is a rare-earth SmCo magnet, 5 mm diameter and 5 mm long. Capacitor 19 is a 680 uF/6.3V surface mount capacitor, 2.8 mm high, from Digikey (www.digikey.com). If a super-capacitor is desired, a 5 mm diameter 0.22 F super-capacitor available from Cooper Electronic Technology (www.cooperet.com), part number BO510-2R5224. The advantage of a super-capacitor is the ability to deliver a very large amount of power for a short time, as may be needed by some applications. A super-capacitor stores between a 100 to a 1000 fold more energy for the same size as a tantalum capacitor. Base 2 and cover 23 (or housing 27) are made of stainless steel, titanium or any other bio-compatible truly hermetic material. A non magnetic material is preferred. Coil 16 is wound with ultra-fine magnet wire such as AWG 56 or 58 available from Wiretron (www.wiretron.com). A prototype device built to these dimensions generated over 100 uW of DC power when operated at a pressure pulse of 100 mmHg, corresponding to being implanted in the left ventricle. Because of the need to maintain a vacuum in the device enclosure for the life of the device, it is important to use construction materials with low outgassing and it is desired to bake the device for a long time and at the maximum temperature allowed before sealing. For example, the device can be baked at 80 deg C. for 100 hours without harming electronic or mechanical components as long as only high temperature polymers are used for internal construction. The exterior, because of the hermetic sealing required, has to be metal with a glass-to-metal seal for the pacing electrode. If a polymer exterior is desired (for example, for hydrophobic outside), it should be applied over the metal.

In the piezoelectric version, generator 5 is a 3 mm wide×25 mm long×0.38 mm thick bimorph, available from Piezo Systems Inc (www.piezo.com), part number T215-A4-103X. The output voltage over ±10V (unloaded) when oscillating at a ±0.5 mm amplitude.

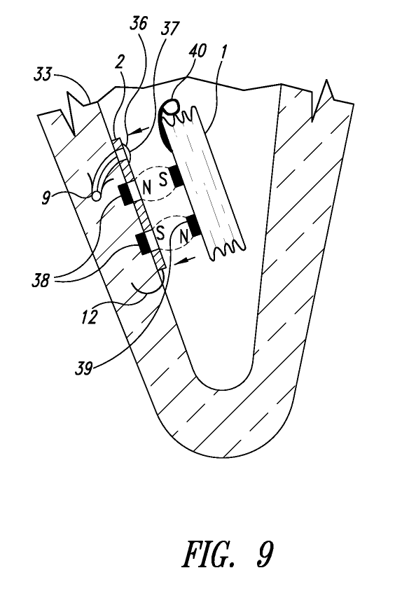

A medical device, for example pacemaker 1c can also have a detachable base, as shown in FIG. 9. Scar tissue may develop around the implanted electrodes 9 and 12, making it difficult to remove a pacemaker 1a, 1b after an extended period, if replacement is needed. A similar difficulty exists today in removal of old pacing leads. By making the base 2 detachable from pacemaker 1c, the base can be left permanently implanted. This also reduces the size of the required catheter, as each part can be introduced into the ventricle separately. Base 2 is equipped with a pair of rare earth recessed disc magnets 38, which are attracted to a similar pair of magnets 39 mounted at the base of pacemaker 1c. Since magnets 39 protrude and magnets 38 are recessed, the two parts snap together and form a rigid joint. The polarity of the magnets is arranged such that they attract only in one orientation, i.e., if the remainder or body of the pacemaker 1c is rotated 180 degrees relative to base 2 the magnets will repel and rotate the remainder of the pacemaker 1cback. Rare-earth magnets as small as 5 mm diameter×1 mm are sufficient. A loop 40 is provided to grab the remainder or body of the pacemaker 1c in case it has to be pulled away from base. To avoid electrolytic corrosion and current leakage, a small silicone rubber pad 36 surrounds electrical contact 37. Contact 37 makes electrical contact with a similar contact (not shown) at the bottom of pacemaker 1. As magnets 38 and 39 attract pacemaker 1c to base 2, silicone seal 36 is compressed to form a water-tight seal.

The small size and percutaneous delivery of the pacemakers 1 allows the implantation of multiple pacemakers 1 into one heart. For example, one unit can be implanted in the left ventricle and one in the right ventricle. The units can be synchronized to generate the optimal pacing sequence (typically the left ventricle unit will pace slightly ahead of the right ventricle) in several different ways:

A. By wireless or inductive communication, using the same methods used today to communicate to the outside world

B. By inductive coupling, made easy due to the close proximity of the units.

C. By sensing the muscle contraction caused by the other pacemaker (or the normal heart operation). As explained earlier, each unit can serve as an accelerometer. The second pacemaker 1 can pace a pre-determined time after sensing the muscle contraction of the first ventricle. If the first pacemaker (or muscle) fails, the second pacemaker 1 will pace after waiting a short delay (a fraction of a second). This arrangement also greatly increases reliability as each pacemaker 1 can take over if no heartbeat is sensed.

While the description is of a pacemaker 1, it is obvious the electricity generated can be used for any other purpose in the body and the device can be installed in, or near, any major artery or rapidly moving organ.

Within the scope of the patent the word “resonance” should be interpreted broadly as any means of extending the motion of the electrical generator beyond the duration of the mechanical excitation, in order to prolong the duration of the current flow into the energy storage device. While resonance is the preferred embodiment, as it is free of wear, other methods can be used to extend the effect of the excitation. For example, a flywheel set in motion by the excitation (using a rack and pinion or a coiled up ribbon) can keep spinning after the excitation ended, thus prolonging the current flow similar to the effect of resonance. Such an arrangement should be considered part of the disclosure. Similarly, the word “motion” in the context of this disclosure should be interpreted as any form of motion: linear, arcuate, rotary, bending, twisting etc.

In one aspect. a method for generating electricity from changes in blood pressure, comprises providing a sealed flexible enclosure: subjecting said enclosure to blood pressure changes and creating relative motion between parts of said enclosure: and using said relative motion to excite a mechanical resonance in an electrical generator.

The generator may operate by changing the magnetic flux in a coil. The generator may be piezoelectric. The enclosure may be at least partially evacuated. The enclosure may be at least partially evacuated and include a compressed spring. The frequency of said resonance is in the range from 10Hz to 100Hz. Generated electricity may be stored in a capacitor.

The method may used in a pacemaker having pacing electrodes, and said electrodes are also used to attach pacemaker to the inside wall of the heart. The pacemaker may be placed in the left ventricle of the heart. The flexible enclosure may comprise a metal bellows.

The method may also include blood pressure sensing.

A plurality of the self powered leadless pacemakers powered by blood pressure changes may have an ability to operate in synchronism.

In another aspect. a method for powering a cardiac pacemaker having an enclosure and a member capable of moving relative to said enclosure. comprises attaching said pacemaker to the heart at an attachment point: creating relative motion between said member and said enclosure: using said relative motion to create an acceleration larger than the acceleration of said attachment point: and using said larger acceleration to excite a mechanical resonance in an electrical generator.

Said member may be inside said enclosure. Said larger acceleration may be created by abruptlv stopping said motion. Said larger acceleration may be created by a snap action incorporated in said motion. The frequency of said resonance may be the range from 10Hz to 100Hz.

A plurality of the self powered leadless pacemakers powered by the relative motion may have an ability to operate in synchronism.

In yet another aspect. a leadless cardiac pacemaker deliverable via a catheter may have a detachable base, said base containing the pacing electrodes. The pacemaker may be held to said detachable base by self-aligning magnets.

1-20. (canceled)

21. An in vivo medical device, comprising:
a flexible enclosure sized to be received in a portion of a cardiovascular system of a human, the flexible enclosure forming an inside that is sealed; and
a resonant generator positioned in the inside of the flexible enclosure, at least a portion of the resonant generator physically coupled to oscillate in response to compression of the flexible enclosure by blood pressure pulses and to produce electrical power in response to the oscillations, a frequency of oscillation of the portion of the resonant generator greater than a frequency of the blood pressure pulses.

22. The medical device of claim 21 wherein the flexible enclosure is a bellows.

23. The medical device of claim 22 wherein the bellows is made of a metal.

24. The medical device of claim 21 wherein the resonant generator includes a piezoelectric element and a mass, the piezoelectric element having a first portion that is fixed and a second portion spaced from the first portion, the mass physically coupled to the piezoelectric element proximate the second portion.

25. The medical device of claim 21 wherein the resonant generator includes a magnet and at least one electrically conductive coil, the magnet mounted for relative movement with respect to the at least one electrically conductive coil.

26. The medical device of claim 21 wherein the resonant generator includes a leaf spring, a magnet, at least one electrically conductive coil, and at least one ferromagnetic sleeve positioned proximate the electrically conductive coil, the magnet mounted to the leaf spring for movement with respect to the at least one electrically conductive coil.

27. The medical device of claim 21 wherein the resonant generator includes a magnet and an electrically conductive coil, the electrically conductive coil mounted for movement with respect to the magnet.

28. The medical device of claim 21 wherein the flexible enclosure is at least a partially evacuated.

29. The medical device of claim 28, further comprising:
at least one spring biasing the flexible enclosure into an uncompressed configuration.

30. The medical device of claim 29 wherein the at least one spring is nonlinear.

31. The medical device of claim 21, further comprising:
a rigid cover physically coupled to seal a first end of the flexible enclosure.

32. The medical device of claim 31, further comprising:
a circuit board physically coupled to seal a second end of the flexible enclosure, the second end opposite the first end of the flexible enclosure.

33. The medical device of claim 21, further comprising:
pacemaker electronics coupled to receive electrical power produced by the resonant generator.

34. The medical device of claim 21, further comprising:
a rectifier coupled to the resonant generator to rectify a current produced by the resonant generator; and
a voltage regulator coupled to the rectifier to adjust a voltage of the rectified current.

35. The medical device of claim 21, further comprising:
an electrical power storage device electrically coupled to receive the electrical power produced by the resonant generator.

36. The medical device of claim 35 wherein the electrical power storage device is a super-capacitor.

37. The medical device of claim 21, further comprising:
a travel limiter structure that limits an amount of travel between portions of the flexible enclosure to compensate for ambient changes in blood pressure.

38. The medical device of claim 21, further comprising:
a computer configured to produce a pulse waveform that is a function of an output of the resonant generator.

39. The medical device of claim 21, further comprising:
a base including at least one anchoring structure configured to physically anchor the base in vivo in the human; and
an attachment structure that detachable couples the flexible enclosure to the base.

40. The medical device of claim 39 wherein the attachment structure includes a first attachment structure fixed to the base.

41. The medical device of claim 39 wherein the attachment structure is a magnetic attachment structure.

42. The medical device of claim 39 wherein the attachment structure includes at least two magnets fixed to the base or a circuit board coupled to the flexible enclosure, and at least two complimentary structures fixed to the base or the circuit board.

43. The medical device of claim 39 wherein the attachment structure is configured to ensure a correct electrical polarity of an electrical coupling made between the base and the resonant generator.

44. The medical device of claim 39 wherein the attachment structure includes a number of retention barbs.

45. The medical device of claim 44 wherein the at least one of the retention barbs is electrically coupled as an electrode to provide electrical current externally from the in vivo medical device to the body.

46. The medical device of claim 39 wherein the flexible enclosure and the base are each sized to be percutaneously delivered individually through the cardiovascular system of the human.

47. The medical device of claim 21 wherein the portion of the resonant generator oscillates at frequencies in a range of between approximately 10Hz and approximately 100Hz.

48. A method of operating a medical device within at least a portion of a body, the method comprising:
transforming oscillation of a portion of a resonant generator that results from movement of an at least partially evacuated flexible enclosure in response to compression of the flexible enclosure by blood pressure pulses into electrical current, where the portion of the resonant generator oscillates at a frequency greater than a frequency of the blood pressure pulses;
rectifying the electrical current; and
supplying the rectified electrical current to a number of electrodes that extend externally from the flexible enclosure within the portion of the body.

49. The method of claim 48, further comprising:
adjusting a voltage of the rectified electrical current before supplying the rectified electrical current to the electrodes.

50. The method of claim 48, further comprising:
temporarily storing the rectified electrical current before supplying the rectified electrical current to the electrodes.

51. The method of claim 48, further comprising:
compensating for relative motion of the flexible enclosure due to ambient changes.

52. A medical device positionable in a body via a catheter, the medical device comprising:
resonant generator means for transforming oscillations of a portion of the resonant generator means that results from movement of an at least partially evacuated flexible enclosure in response to compression of the flexible enclosure by blood pressure pulses into electrical current, where the portion of the resonant generator oscillates at a frequency greater than a frequency of the blood pressure pulses;
a rectifier electrically coupled to rectify the electrical current; and
a number of electrodes that extend externally from the flexible enclosure within the portion of the body, at least one of the electrodes electrically coupled to supply the rectified electrical current to the body.

53. The medical device of claim 52, further comprising:
means for temporarily storing the rectified current electrically coupled to the rectifier.

54. The medical device of claim 52, further comprising:
means for compensating for ambient changes.

55. The medical device of claim 52, further comprising:
means for producing a pulse waveform based on a characteristic of the electrical current.

56. A medical device, comprising:
an enclosure sized to be positioned percutaneously via a cardiovascular system of a human, the enclosure forming an inside; and
a resonant generator, at least a portion of the resonant generator physically coupled to resonantly oscillate in response to movement imparted to the enclosure directly by at least a portion of a heart muscle of the human and to produce electrical power in response to the resonant oscillations, at least the portion of the resonant generator mounted in the inside of the enclosure such that an acceleration of the portion of the resonant generator is greater than an acceleration of the portion of the heat muscle and a frequency of oscillation of the portion of the resonant generator is greater than the frequency of the movement of the portion of the heart muscle.

57. The medical device of claim 56 wherein the portion of the resonant generator oscillates at frequencies in a range of between approximately 10Hz and approximately 100Hz.

58. The medical device of claim 56, further comprising:
a frame mounted in the enclosure to oscillate with respect thereto, wherein at least the portion of the resonant generator is mounted to the frame.

59. The medical device of claim 58, further comprising:
a number of stops positioned in the enclosure to abruptly limit the oscillation of the frame.

60. The medical device of claim 59, further comprising:
a first spring that couples a first end of the frame to the enclosure.

61. The medical device of claim 60, further comprising:
a second spring that couples a second end the frame to the enclosure, the second end of the frame spaced from the first end of the frame.

62. The medical device of claim 60 wherein the resonant generator includes a piezoelectric element and a mass, the piezoelectric element having a first portion coupled to the frame and a second portion coupled to the mass.

63. The medical device of claim 60 wherein the resonant generator includes a leaf spring, a magnet, at least one electrically conductive coil, and at least one ferromagnetic sleeve positioned proximate the electrically conductive coil, the magnet mounted to the leaf spring for movement with respect to the at least one electrically conductive coil and the leaf spring coupled to the frame.

64. The medical device of claim 56, further comprising:
a circuit board received in the enclosure.

65. The medical device of claim 56, further comprising:
pacemaker electronics carried in the enclosure and coupled to receive electrical power produced by the resonant generator.

66. The medical device of claim 56, further comprising:
a rectifier coupled to the resonant generator to rectify a current produced by the resonant generator; and
a voltage regulator coupled to the rectifier to adjust a voltage of the rectified current.

67. The medical device of claim 56, further comprising:
an electrical power storage device electrically coupled to receive electrical power produced by the resonant generator.

68. The medical device of claim 67 wherein the electrical power storage device is a super-capacitor.

69. The medical device of claim 56, further comprising:
a computer configured to produce a pulse waveform that is a function of an output of the resonant generator.

70. The medical device of claim 56, further comprising:
a base including at least one anchoring structure configured to physically anchor the base in vivo in the human; and
an attachment structure that detachable couples the enclosure to the base.

71. The medical device of claim 70 wherein the attachment structure includes a first attachment structure fixed to the base.

72. The medical device of claim 70 wherein the attachment structure is a magnetic attachment structure.

73. The medical device of claim 70 wherein the attachment structure includes at least two magnets fixed to the base or a circuit board coupled to the enclosure, and at least two complimentary structures fixed to the base or the circuit board.

74. The medical device of claim 70 wherein the attachment structure is configured to ensure a correct electrical polarity of an electrical coupling made with the base.

75. The medical device of claim 70 wherein the attachment structure includes a number of retention barbs.

76. The medical device of claim 75 wherein the at least one of the retention barbs is electrically coupled as an electrode to provide electrical current externally from the in vivo medical device to the body.

77. The medical device of claim 70 wherein the enclosure and the base are each sized to be percutaneously delivered individually through the cardiovascular system of the human.

78. A method of operating a medical device within at least a portion of a body, the method comprising:
transforming oscillation of a portion of a resonant generator that results from movement imparted to an enclosure directly by movement of at least a portion of a heart muscle of the body into electrical current where a resulting acceleration of the portion of the resonant generator is greater than an acceleration of the portion of the heat muscle and a frequency of oscillation of the portion of the resonant generator is greater than the frequency of the movement of the portion of the heart muscle;
rectifying the electrical current; and
supplying the rectified electrical current to a number of electrodes that extend externally from the enclosure within the portion of the body.

79. The method of claim 78, further comprising:
adjusting a voltage of the rectified electrical current before supplying the rectified electrical current to the body via a number of electrodes.

80. The method of claim 78, further comprising:
temporarily storing the rectified electrical current before supplying the rectified electrical current to the body via a number of electrodes.

81. The method of claim 78 wherein the enclosure is flexible, and further comprising:
compensating for relative motion of the enclosure caused by changes in ambient conditions.

82. A medical device positionable in a body via a catheter, the medical device comprising:
an enclosure;
a resonant generator means for transforming oscillation of a portion of a resonant generator that results from movement imparted to an enclosure directly by movement of at least a portion of a heart muscle of the body into electrical current where a resulting acceleration of the portion of the resonant generator is greater than an acceleration of the portion of the heat muscle and a frequency of oscillation of the portion of the resonant generator is greater than the frequency of the movement of the portion of the heart muscle;
a rectifier electrically coupled to rectify the electrical current; and
a number of electrodes that extend externally from the enclosure within the portion of the body electrically coupled to supply the rectified electrical current to the body.

83. The medical device of claim 82 wherein the resonant generator means includes a frame mounted in the enclosure to oscillate with respect thereto, wherein at least the portion of the resonant generator is mounted to the frame and a number of stops positioned to abruptly limit the oscillation of the frame.

84. The medical device of claim 83 wherein the resonant generator means includes a first spring that oscillatingly couples a first end of the frame to the enclosure.

85. The medical device of claim 84 wherein the resonant generator means includes a second spring that oscillatingly couples a second end the frame to the enclosure, the second end of the frame spaced from the first end of the frame.

86. The medical device of claim 85 wherein the resonant generator means includes a piezoelectric element and a mass, the piezoelectric element having a first portion coupled to the frame and a second portion coupled to the mass.

87. The medical device of claim 85 wherein the resonant generator means includes a leaf spring, a magnet, at least one electrically conductive coil, and at least one ferromagnetic sleeve positioned proximate the electrically conductive coil, the magnet mounted to the leaf spring for movement with respect to the at least one electrically conductive coil and the leaf spring coupled to the frame.

88. The medical device of claim 82, further comprising:
means for temporarily storing the rectified current electrically coupled to the rectifier.

89. The medical device of claim 82, further comprising:
means for producing a pulse waveform that is a function of the electrical current.

90. An in vivo medical device, comprising:
a base including at least one anchoring structure configured to physically anchor the base in vivo in a human;
an enclosure sized to be percutaneously delivered through a cardiovascular system of the human;
an attachment structure that detachably attaches the enclosure to the base; and
a generator received in the flexible enclosure and physically mounted to transform mechanical movement into electrical power.

91. The medical device of claim 90 wherein the generator is a resonant generator that includes a piezoelectric element and a mass, the piezoelectric element having a first portion coupled to the base and a second portion coupled to the mass.

92. The medical device of claim 90 wherein the generator is a resonant generator that includes a magnet and at least one electrically conductive coil, the magnet mounted for relative movement with respect to the at least one electrically conductive coil.

93. The medical device of claim 90 wherein the generator is a resonant generator that includes a leaf spring, a magnet, at least one electrically conductive coil, and at least one ferromagnetic sleeve positioned proximate the electrically conductive coil, the magnet mounted to the leaf spring for movement with respect to the at least one electrically conductive coil.

94. The medical device of claim 90, further comprising:
a frame mounted in the enclosure to oscillate with respect thereto, wherein at least the portion of the resonant generator is mounted to the frame and a number of stops positioned to abruptly limit the oscillation of the frame.

95. The medical device of claim 94, further comprising:
a first spring that couples a first end of the frame to the enclosure.

96. The medical device of claim 95, further comprising:
a second spring that couples a second end the frame to the enclosure, the second end of the frame spaced from the first end of the frame.

97. The medical device of claim 90 wherein the generator is a resonant generator that has a portion that oscillates at frequencies in a range of between approximately 10Hz and approximately 100Hz.

98. The medical device of claim 90 wherein the enclosure is a flexible enclosure.

99. The medical device of claim 98 wherein the flexible enclosure is a bellows.

100. The medical device of claim 99 wherein the bellows is made of a metal.

101. The medical device of claim 98 wherein the flexible enclosure is at least partially evacuated, and further comprising:
at least one spring positioned in the inside of the flexible enclosure, the at least one spring biasing the flexible enclosure into an uncompressed configuration.

102. The medical device of claim 101 wherein the at least one spring is nonlinear.

103. The medical device of claim 101, further comprising:
a rigid cover physically coupled to seal a first end of the flexible enclosure.

104. The medical device of claim 103, further comprising:
a circuit board physically coupled to seal a second end of the flexible enclosure, the second end opposite the first end of the flexible enclosure.

105. The medical device of claim 101, further comprising:
a travel limiter structure that limits an amount of travel between the portions of the flexible enclosure to compensate for ambient changes in blood pressure.

106. The medical device of claim 90, further comprising:
pacemaker electronics received in the enclosure and coupled to receive electrical power produced by the generator.

107. The medical device of claim 90, further comprising:
a rectifier coupled to the generator to rectify a current produced by the resonant generator; and
a voltage regulator coupled to the rectifier to adjust a voltage of the rectified current.

108. The medical device of claim 90, further comprising:
an electrical power storage device electrically coupled to receive an electrical current produced by the generator.

109. The medical device of claim 108 wherein the electrical power storage device is a super-capacitor.

110. The medical device of claim 90, further comprising:
a computer configured to produce a pulse waveform that is a function of an output of the generator.

111. The medical device of claim 90 wherein the attachment structure includes a magnetic attachment structure.

112. The medical device of claim 90 wherein the attachment structure includes at least two magnets fixed to the base or a circuit board coupled to the flexible enclosure, and at least two complimentary structures fixed to the base or the circuit board.

113. The medical device of claim 90 wherein the attachment structure is configured to ensure a correct electrical polarity of an electrical coupling made with the base.

114. The medical device of claim 90 wherein the attachment structure includes a number of retention barbs.

115. The medical device of claim 114 wherein at least one of the retention barbs is electrically coupled as an electrode to provide electrical current externally from the in vivo medical device to the body.

116. The medical device of claim 90 wherein the base is sized to be percutaneously delivered individually through the cardiovascular system of the human.

117. The medical device of claim 90, further comprising:
a retrieval loop fixedly coupled to the enclosure to allow percutaneous retrieval of the enclosure from the base.

118. A method of operating a medical device within at least a portion of a body, the method comprising:
transforming mechanical movement into an electrical current by a generator located in an enclosure and carried by a circuit board;
rectifying the electrical current; and
supplying the rectified current to a detachable base to which the circuit board is detachably coupled, the detachable base anchored within the portion of the body.

119. The method of claim 118, further comprising:
supplying the rectified electrical current to a number of electrodes that extend externally from the detachable base and which anchor the detachable base within the portion of the body.

120. The method of claim 118, further comprising:
adjusting a voltage of the rectified electrical current before supplying the rectified electrical current to the detachable base.

121. The method of claim 118, further comprising:
temporarily storing the rectified electrical current before supplying the rectified electrical current to the detachable base.

122. The method of claim 118 wherein the enclosure is a flexible enclosure, and further comprising:
compensating for relative motion of the enclosure caused by changes in ambient conditions.

123. A medical device positionable in a body via a catheter, the medical device comprising:
enclosure means for providing a sealed inside;
generator means for transforming mechanical movement into an electrical current; base means for providing a base anchored in side a portion of a human body; and attachment means for detachably attaching the enclosure to the base means.

124. The medical device of claim 123 wherein in the generator means is a resonate generator including at least one portion that oscillates.

125. The medical device of claim 124 wherein the resonant generator includes a frame and a number of stops, the frame mounted in the enclosure to oscillate with respect thereto, wherein at least the portion of the resonant generator is mounted to the frame and the stops are positioned to abruptly limit the oscillation of the frame.

126. The medical device of claim 125 wherein the resonant generator further includes a first spring that oscillatingly couples a first end of the frame to the enclosure.

127. The medical device of claim 126 wherein the resonant generator includes a second spring that oscillatingly couples a second end the frame to the enclosure, the second end of the frame spaced from the first end of the frame.

128. The medical device of claim 125 wherein the resonant generator includes a piezoelectric element and a mass, the piezoelectric element having a first portion coupled to the frame and a second portion coupled to the mass.

129. The medical device of claim 125 wherein the resonant generator includes a leaf spring, a magnet, at least one electrically conductive coil, and at least one ferromagnetic sleeve positioned proximate the electrically conductive coil, the magnet mounted to the leaf spring for movement with respect to the at least one electrically conductive coil and the leaf spring coupled to the frame.

130. The medical device of claim 124 wherein the portion of the resonant generator oscillates in resonance at frequencies in a range of between approximately 10Hz and approximately 100Hz.

131. The medical device of claim 123, further comprising:
a rectifier electrically coupled to rectify the electrical current.

132. The medical device of claim 131, further comprising:
a number of electrodes that extend externally from the base means to make electrical contact with the body.

133. The medical device of claim 132, further comprising:
means for transferring the rectified electrical current to the number of electrodes.

134. The medical device of claim 131, further comprising:
means for temporarily storing the rectified electrical current electrically coupled to the rectifier.

135. The medical device of claim 123 wherein the attachment means includes a number of magnets.

136. The medical device of claim 123 wherein the attachment means is configured to ensure a correct electrical polarity between the generator means and the base means.
